(19) United States
(12) Patent Application Publication
(10) Pub. No.: US 2019/0369020 A1
(43) Pub. Date: Dec. 5, 2019

(54) METHOD FOR PREDICTING MATERIAL PERFORMANCE OF POLYIMIDE MATERIAL

(71) Applicant: SABIC GLOBAL TECHNOLOGIES B.V., BERGEN OP ZOOM (NL)

(72) Inventors: Bing Zhou, Mt Vernon, IN (US); Sasi S. Kannamkumarath, Mt Vernon, IN (US); Daniel F. Lowery, Mt Vernon, IN (US)

(21) Appl. No.: 16/332,146

(22) PCT Filed: Sep. 13, 2017
(86) PCT No.: PCT/US2017/051327

§ 371 Date: Mar. 11, 2019

Related U.S. Application Data
(60) Provisional application No. 62/393,709, filed on Sep. 13, 2016.

(51) Int. Cl. G01N 21/59; G01N 1/38; C08J 11/04; C08G 73/10; G16C 20/10

(52) U.S. Cl. CPC G01N 21/59; G01N 1/38; C08J 11/04; C08J 2379/08; C08G 73/1028; C08G 73/1064; G16C 20/10; C08G 73/1046

(57) ABSTRACT

A method for predicting the material performance of a polyimide, the method comprising: dissolving a sample of the polyimide in a solvent to form a polyimide solution; determining a transmittance of the polyimide solution at a wavelength of 400 to 800 nm, preferably at a wavelength of 450 to 600 nm to obtain a test value; inputting the test value into a predetermined prediction equation to obtain a predicted material performance value; and determining if the predicted material performance value is within a desired range of material performance.

### CROSS-REFERENCE TO RELATED APPLICATIONS

This applications claims priority to U.S. provisional application Ser. No. 62/393,709, filed Sep. 13, 2016, the content of which is hereby incorporated by reference.

### BACKGROUND

Polyimides, in particular polyetherimides (PEI) are amorphous, transparent, high performance polymers having a glass transition temperature (Tg) of greater than 180° C. Polyetherimides further have high strength, toughness, heat resistance, and modulus, and broad chemical resistance, and so are widely used in industries as diverse as automotive, telecommunication, aerospace, electrical/electronics, transportation, and healthcare. Polyetherimides have shown versatility in various manufacturing processes, proving amenable to techniques including injection molding, extrusion, and thermoforming, to prepare various articles. Although polyimides can possess beneficial properties, polyimides can have an amber color which can be undesirable, or result in undesirable properties for certain applications. It can be difficult to accurately and reproducibly determine the color of molded materials.

There accordingly is a need for a method for predicting the color of polyimides.

### SUMMARY

A method for predicting the material performance of a polyimide, the method comprising: dissolving a sample of the polyimide in a solvent to form a polyimide solution; determining a transmittance of the polyimide solution at a wavelength of 400 to 800 nm, preferably at a wavelength of 450 to 600 nm to obtain a test value; inputting the test value into a predetermined prediction equation to obtain a predicted material performance value; and determining if the predicted material performance value is within a desired range of material performance is provided.

A method of manufacturing an article from a polyimide, the method comprising: predicting the material performance of the polyimide in accordance with a method provided; and proceeding with manufacture of the predicted material performance value is within a desired range of material performance.

The above described and other features are exemplified by the following detailed description.

### BRIEF DESCRIPTION OF THE DRAWINGS

The following figures are exemplary embodiments.

FIG. 1A-1K show transmittance versus grayscale at wavelengths from 450 nanometers (nm) to 950 nm at intervals of 50 nm.

FIG. 2A shows coefficients of determination between absorbance and grayscale at wavelengths from 450 nm to 950 nm at intervals of 50 nm.

FIG. 2B shows coefficients of determination between absorbance and grayscale at wavelengths from 450 nm to 550 nm at intervals of 10 nm.

FIG. 3 shows a scatterplot of calculated grayscale values versus measured grayscale values.

### DETAILED DESCRIPTION

Evaluation of the color, including yellowness index, and transmittance of polyimides, including polyetherimides, is difficult due to the amount of material needed to mold a plaque (around 5 pounds) and poor repeatability of the measurements. The complex manufacturing process of polyimides also results in lot-to-lot variations in the yellowness index and transmittance. Some users of polyimide materials use evaluation methods other than yellowness index and transmittance to evaluate the suitability of materials for an end use. One of these evaluation methods is a measurement of the grayscale of the material. Grayscale is a way of representing color with an integer. Since grayscale is a method used to evaluate suitability of materials, it would be useful to be able to predict the grayscale value of a sample using transmittance or yellowness index.

The inventors hereof have discovered a method and system to predict a performance value of a polyimide by dissolving a sample of the polyimide in a solvent, determining a transmittance of the polyimide, and using the transmittance value to obtain a predicted performance value. The predicted performance value can be used to determine if the predicted material performance is within a desired range of material performance. The transmittance value can be input into a predetermined prediction equation to obtain a predicted performance value.

The predetermined prediction equation is determined by obtaining a performance value for a polyimide sample; obtaining a transmittance value for the polyimide sample; calculating a mathematical relationship between the performance value and the transmittance value to obtain a predetermined prediction equation. The performance value can be, for example, related to an absorbance property, a transmittance property, a color property, an optical property, an aesthetic property, or a combination comprising at least one of the foregoing. In an embodiment, the performance value is related to a color property.

The polyimide can be any one of a number of polyimides, including polyetherimide. The polyetherimide can be a polyetherimide homopolymer, a polyetherimide copolymer, or a combination comprising at least one of the foregoing. In an embodiment, the polyimide is a polyetherimide comprising bisphenol-A dianhydride and diamino diphenyl sulfone monomers.

The solvent used to dissolve the polyimide can be N-methyl-2-pyrrolidone (NMP), dichloromethane, chloroform, dimethyl sulfoxide (DMSO), tetrahydrofuran (THF), dimethylacetamide (DMAcAm), N,N-dimethylformamide (DMF), hexafluoroisopropanol (HFIP), cresol, or a combination comprising at least one of the foregoing, and other solvents capable of dissolving polyimide, including polyetherimide.

The polyimide solution can be at any suitable concentration to provide the desired transmittance value. In an embodiment, the polyimide solution is at a concentration between 0.001 grams/milliliter (g/mL) to 1 g/mL, preferably 0.05 g/mL to 0.2 g/mL. In an embodiment, the polyimide solution is at a concentration of 0.05 to 0.15 g/mL.

The methods provided here allow for the use of a smaller required sample size for dissolution in the solvent than making a molded material, for example a plaque or color chip. In an embodiment, the sample size is 0.01 grams to 2 kilograms of the polyetherimide, preferably the sample size is 0.5 grams to 100 grams of the polyetherimide.

The methods described here can be used to reject a polyimide if the predicted material performance value is not within a desired range of material performance. For example, if the material performance is a color property, the polyimide can be rejected if the predicted material performance value is not within ±10 percent, preferably ±5 percent, more preferably ±1 percent, or a desired range of the color property. In an embodiment, the color property is a grayscale value. In an embodiment, the color property is a yellowness value.

The methods described here can also be used to accept a polyimide if the predicted material performance value is within a desired range of material performance.

In some embodiments the polyimide can be a polyetherimide, preferably a polyetherimide comprising units derived from the reaction of bisphenol A dianhydride and m-phenylene diamine. The polyimide can be a polyetherimide homopolymer, a polyetherimide co-polymer such as a poly(etherimide sulfone).

Polyimides comprise more than 1, for example 10 to 1000, or 10 to 500, structural units of formula (1)

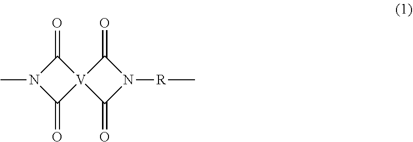

wherein each V is the same or different, and is a substituted or unsubstituted tetravalent C₄₋₄₀ hydrocarbon group, for example a substituted or unsubstituted C₆₋₂₀ aromatic hydrocarbon group, a substituted or unsubstituted, straight or branched chain, saturated or unsaturated C₂₋₂₀ aliphatic group, or a substituted or unsubstituted C₄₋₈ cycloalkylene group or a halogenated derivative thereof, in particular a substituted or unsubstituted C₆₋₂₀ aromatic hydrocarbon group. Exemplary aromatic hydrocarbon groups include any of those of the formulas

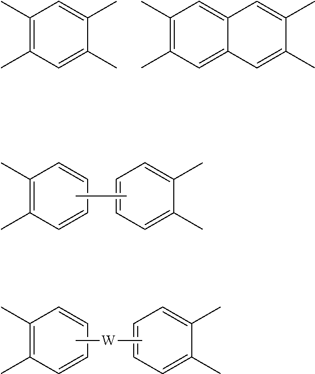

wherein W is —O—, —S—, —C(O)—, —SO₂—, —SO—, —C_{y}H_{2y}— wherein y is an integer from 1 to 5 or a halogenated derivative thereof (which includes perfluoroalkylene groups), or a group of the formula T as described in formula (3) below.

Each R in formula (1) is the same or different, and is a substituted or unsubstituted divalent organic group, such as a C₆₋₂₀ aromatic hydrocarbon group or a halogenated derivative thereof, a straight or branched chain C₂₋₂₀ alkylene group or a halogenated derivative thereof, a C₃₋₈ cycloalkylene group or halogenated derivative thereof, in particular a divalent group of formulas (2)

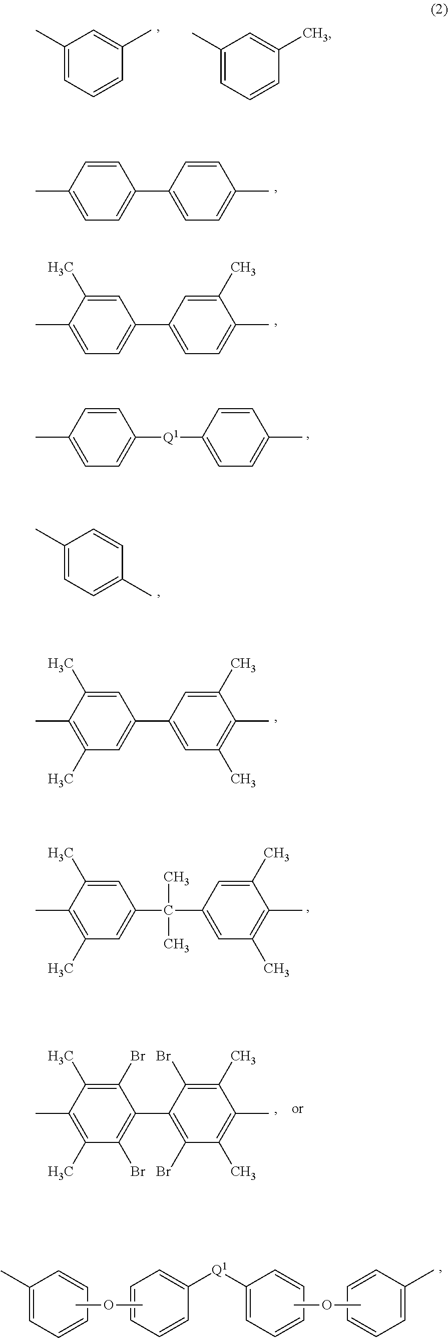

wherein Q′ is —O—, —S—, —C(O)—, —SO₂—, —SO—, —C_{y}H_{2y}— wherein y is an integer from 1 to 5 or a halogenated derivative thereof (which includes perfluoroalkylene groups), or —(C₆H₁₀)_{z}— wherein z is an integer from 1 to 4. In an embodiment R is m-phenylene, p-phenylene, or a diaryl sulfone, e.g., bis(p,p-diphenylene) sulfone.

Polyetherimides are a class of polyimides that comprise more than 1, for example 10 to 1000, or 10 to 500, structural units of formula (3)

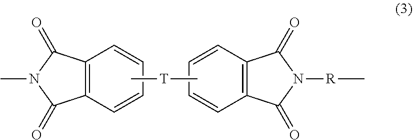

wherein each R is the same or different, and is as described in formula (1).

Further in formula (3), T is —O— or a group of the formula —O—Z—O— wherein the divalent bonds of the —O— or the —O—Z—O— group are in the 3,3′, 3,4′, 4,3′, or the 4,4′ positions. The group Z in —O—Z—O— of formula (3) is also a substituted or unsubstituted divalent organic group, and can be an aromatic C₆₋₂₄ monocyclic or polycyclic moiety optionally substituted with 1 to 6 C₁₋₈ alkyl groups, 1 to 8 halogen atoms, or a combination thereof, provided that the valence of Z is not exceeded. Exemplary groups Z include groups derived from a dihydroxy compound of formula (4)

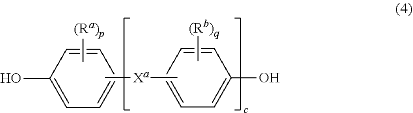

wherein R^{a} and R^{b} can be the same or different and are a halogen atom or a monovalent C₁₋₆ alkyl group, for example; p and q are each independently integers of 0 to 4; c is 0 to 4; and X^{a} is a bridging group connecting the hydroxy-substituted aromatic groups, where the bridging group and the hydroxy substituent of each C₆ arylene group are disposed ortho, meta, or para (specifically para) to each other on the C₆ arylene group. The bridging group X^{a} can be a single bond, —O—, —S—, —S(O)—, —S(O)₂—, —C(O)—, or a C₁₋₁₈ organic bridging group. The C₁₋₁₈ organic bridging group can be cyclic or acyclic, aromatic or non-aromatic, and can further comprise heteroatoms such as halogens, oxygen, nitrogen, sulfur, silicon, or phosphorous. The C₁₋₁₈ organic group can be disposed such that the C₆ arylene groups connected thereto are each connected to a common alkylidene carbon or to different carbons of the C₁₋₁₈ organic bridging group. A specific example of a group Z is a divalent group of formula (4a)

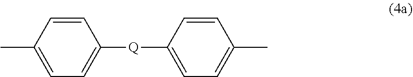

wherein Q is —O—, —S—, —C(O)—, —SO₂—, —SO—, or —C_{y}H_{2y}— wherein y is an integer from 1 to 5 or a halogenated derivative thereof (including a perfluoroalkylene group). In a specific embodiment Z is a derived from bisphenol A, such that Q in formula (4a) is 2,2-isopropylidene.

In an embodiment in formula (3), R is m-phenylene or p-phenylene, or a combination comprising at least one of the foregoing, and T is —O—Z—O— wherein Z is a divalent group of formula (2). Alternatively, R is m-phenylene, p-phenylene, or a combination comprising at least one of the foregoing, and T is —O—Z—O wherein Z is a divalent group of formula (4a) and Q is 2,2-isopropylidene. Alternatively, the polyetherimide can be a copolymer comprising additional structural polyetherimide units of formula (1) wherein at least 50 mole percent (mol %) of the R groups are bis(3,4′-phenylene)sulfone, bis(3,3′-phenylene)sulfone, or a combination comprising at least one of the foregoing and the remaining R groups are p-phenylene, m-phenylene or a combination comprising at least one of the foregoing; and Z is 2,2-(4-phenylene)isopropylidene, i.e., a bisphenol A moiety.

The polyetherimide copolymer optionally comprises additional structural imide units, for example imide units of formula (1) wherein R is as described in formula (1) and V is a linker of the formulas

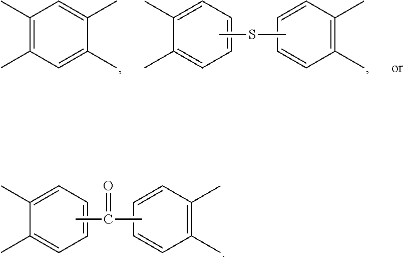

These additional structural imide units can be present in amounts from 0 to 10 mole % of the total number of units, specifically 0 to 5 mole %, more specifically 0 to 2 mole %. In an embodiment no additional imide units are present in the polyetherimide.

The polyimide and polyetherimide can be prepared by any of the methods well known to those skilled in the art, including the reaction of an aromatic bis(ether anhydride) of formula (5a) or formula (5b)

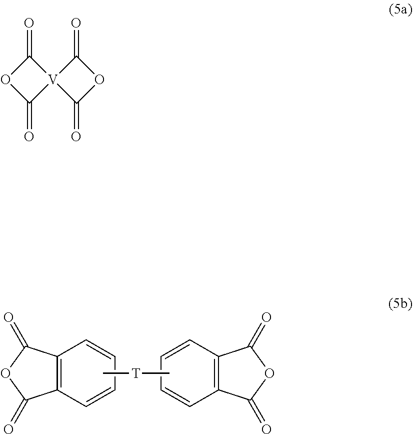

or a chemical equivalent thereof, with an organic diamine of formula (6)

H₂N—R—NH₂ (6)

wherein V, T, and R are defined as described above. Copolymers of the polyetherimides can be manufactured using a combination of an aromatic bis(ether anhydride) of formula (5a) or (5b) and a different bis(anhydride), for example a bis(anhydride) wherein T does not contain an ether functionality, for example T is a sulfone.

Illustrative examples of bis(anhydride)s include 3,3-bis[4-(3,4-dicarboxyphenoxy)phenyl]propane dianhydride; 4,4′-bis(3,4-dicarboxyphenoxy)diphenyl ether dianhydride; 4,4′-bis(3,4-dicarboxyphenoxy)diphenyl sulfide dianhydride; 4,4′-bis(3,4-dicarboxyphenoxy)benzophenone dianhydride; 4,4′-bis(3,4-dicarboxyphenoxy)diphenyl sulfone dianhydride; 2,2-bis[4-(2,3-dicarboxyphenoxy)phenyl]propane dianhydride; 4,4′-bis(2,3-dicarboxyphenoxy)diphenyl ether dianhydride; 4,4′-bis(2,3-dicarboxyphenoxy)diphenyl sulfide dianhydride; 4,4′-bis(2,3-dicarboxyphenoxy)benzophenone dianhydride; 4,4′-bis(2,3-dicarboxyphenoxy)diphenyl sulfone dianhydride; 4-(2,3-dicarboxyphenoxy)-4′-(3,4-dicarboxyphenoxy)diphenyl-2,2-propane dianhydride; 4-(2,3-dicarboxyphenoxy)-4′-(3,4-dicarboxyphenoxy)diphenyl ether dianhydride; 4-(2,3-dicarboxyphenoxy)-4′-(3,4-dicarboxyphenoxy)diphenyl sulfide dianhydride; 4-(2,3-dicarboxyphenoxy)-4′-(3,4-dicarboxyphenoxy)benzophenone dianhydride; and, 4-(2,3-dicarboxyphenoxy)-4′-(3,4-dicarboxyphenoxy)diphenyl sulfone dianhydride, as well as various combinations thereof.

Examples of organic diamines include ethylenediamine, propylenediamine, trimethylenediamine, diethylenetriamine, triethylene tetramine, hexamethylenediamine, heptamethylenediamine, octamethylenediamine, nonamethylenediamine, decamethylenediamine, 1,12-dodecanediamine, 1,18-octadecanediamine, 3-methylheptamethylenediamine, 4,4-dimethylheptamethylenediamine, 4-methylnonamethylenediamine, 5-methylnonamethylenediamine, 2,5-dimethylhexamethylenediamine, 2,5-dimethylheptamethylenediamine, 2,2-dimethylpropylenediamine, N-methyl-bis (3-aminopropyl) amine, 3-methoxyhexamethylenediamine, 1,2-bis(3-aminopropoxy) ethane, bis(3-aminopropyl) sulfide, 1,4-cyclohexanediamine, bis-(4-aminocyclohexyl) methane, m-phenylenediamine, p-phenylenediamine, 2,4-diaminotoluene, 2,6-diaminotoluene, m-xylylenediamine, p-xylylenediamine, 2-methyl-4,6-diethyl-1,3-phenylene-diamine, 5-methyl-4,6-diethyl-1,3-phenylene-diamine, benzidine, 3,3′-dimethylbenzidine, 3,3′-dimethoxybenzidine, 1,5-diaminonaphthalene, bis(4-aminophenyl) methane, bis(2-chloro-4-amino-3,5-diethylphenyl) methane, bis(4-aminophenyl) propane, 2,4-bis(p-amino-t-butyl) toluene, bis(p-amino-t-butylphenyl) ether, bis(p-methyl-o-aminophenyl) benzene, bis(p-methyl-o-aminopentyl) benzene, 1,3-diamino-4-isopropylbenzene, bis(4-aminophenyl) sulfide, bis-(4-aminophenyl) sulfone, and bis(4-aminophenyl) ether. Combinations of these compounds can also be used. In some embodiments the organic diamine is m-phenylenediamine, p-phenylenediamine, sulfonyl dianiline, or a combination including at least one of the foregoing.

Other methods for the manufacture of polyimides and polyetherimides are known, and can include residues derived from chemical equivalents of the foregoing anhydrides and diamines, e.g., residues of bisphenol A, p-phenylenediamine, m-phenylene diamine, bis(p-phenyleneamino) sulfone, or a combination comprising at least one of the foregoing.

The polyimide can also comprise a polyetherimide sulfone copolymer comprising polyetherimide units of formula (1) and sulfone units, wherein at least 50 mole % of the linkers V and the groups R in formula (1) comprise a divalent arylene sulfone group. For example, all linkers V, but no groups R, can contain an arylene sulfone group; or all groups R but no linkers V can contain an arylene sulfone group; or an arylene sulfone can be present in some fraction of the linkers V and R groups, provided that the total mole fraction of V and R groups containing an aryl sulfone group is greater than or equal to 50 mole %.

It is to be understood that polyetherimides, including polyether sulfones, can optionally comprise linkers V that do not contain ether groups or ether and sulfone groups, for example linkers of formula (7):

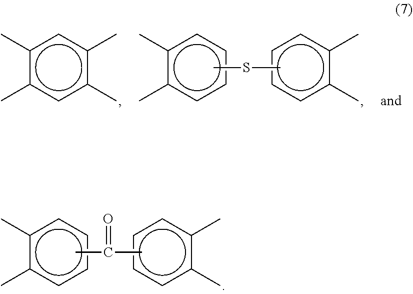

Imide units containing such linkers are generally be present in amounts ranging from 0 to 10 mole % of the total number of units, specifically 0 to 5 mole %. In one embodiment no additional linkers V are present in the polyetherimides and polyetherimide sulfones.

In another specific embodiment, the polyetherimide sulfone contains 10 to 500 structural units of formula (6).

The polyetherimide sulfones can be prepared by various methods, including, but not limited to, the reaction of a bis(phthalimide) of formula (8):

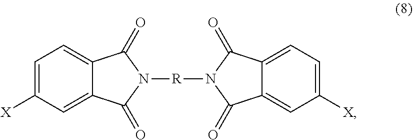

wherein R is as described above and X is a nitro group or a halogen. Bis-phthalimides (8) can be formed, for example, by the condensation of the corresponding anhydride of formula (9):

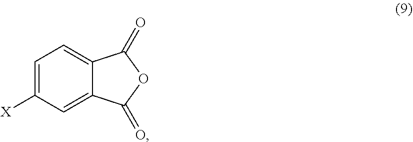

wherein X is a nitro group or halogen, with an organic diamine of the formula (10):

H₂N—R—NH₂ (10),

wherein R is as described above, containing a sulfone group. Mixtures of these amines can be used. Illustrative examples of amine compounds of formula (10) containing sulfone groups include but are not limited to, diamino diphenyl sulfone (DDS) and bis(aminophenoxy phenyl) sulfones (BAPS). Combinations comprising any of the foregoing amines can be used.

The polyimides, specifically the polyetherimides, can have a melt index of 0.1 to 10 grams per minute (g/min), as measured by American Society for Testing Materials (ASTM) D1238 at 340 to 370° C., using a 6.7 kilogram (kg) weight. In some embodiments, the polyetherimide polymer has a weight average molecular weight (Mw) of 1,000 to 150,000 grams/mole (Dalton), as measured by gel permeation chromatography, using polystyrene standards. In some embodiments the polyetherimide has an Mw of 10,000 to 80,000 Daltons. Such polyetherimide polymers typically have an intrinsic viscosity greater than 0.2 deciliters per gram (dl/g), or, more specifically, 0.35 to 0.7 dl/g as measured in m-cresol at 25° C. The polyimides can have a glass transition temperature of greater than 180° C., specifically of 200° C. to 500° C., as measured using differential scanning calorimetry (DSC) per ASTM test D3418. In some embodiments, the polyimide and, in particular, a polyetherimide has a glass transition temperature of 240 to 350° C.

The methods described herein are further illustrated by the following non-limiting examples.

### EXAMPLES

TABLE 1

| Component | Description | Supplier |
|---|---|---|
| NMP | N-Methyl-2-pyrrolidone | |
| PEI | Polyetherimide made from the reaction of | SABIC |
| | bisphenol A dianhydride with | |
| | diaminodiphenylsulfone, having | |
| | a glass transition temperature of | |
| | 267° C. (EXTEM XH1015) | |

PEI samples for testing were used in different forms, including virgin pellets, regrind pellets, molded parts, color plaques, powder, and foam.

Yellowness index (YI) was obtained per ASTM method D1925 (displayed as TI D1925) on an X-rite ColorEye 7000A with specular component and UV light included and calculated for D65 illumination.

N-Methyl-2-pyrrolidone (NMP) was identified as a suitable solvent for dissolving the polyetherimide samples and conducting optical measurements in solution. Heating NMP solvent to 100° C. or using sonication significantly reduced the PEI dissolution time compared to passive manual shaking or using a wrist shaker. Most of the samples of PEI could be dissolved within 1 to 2 hours by heating or sonicating. A balance with 4 or 5 decimal place accuracy was used for all the weight measurements. A dispenser from an auto-titrator unit (Metrohm 901) was used to dispense 25 milliliters (mL) solvent into the sample bottle accurately and precisely to improve repeatability and reproducibility of sample dissolution. The maximum permissible error allowed for these experiments was less than 40 microliters (μL). The repeatability and reproducibility of sample dissolution was improved by elimination of manually measuring and transferring the solvents.

Eight pellet samples were dissolved in NMP as described above. Color chips were also molded from these samples and the absorbance/transmittance was compared to yellowness index of the molded color chips, as described below. After the sample preparation and optimization of the instrumental setting of the Perkin Elmer Lambda 950 UV-Vis-NIR spectrometer were performed, absorbance/transmission data were collected at 950 nm and 850 nm for the samples. The results are provided in Table 2.

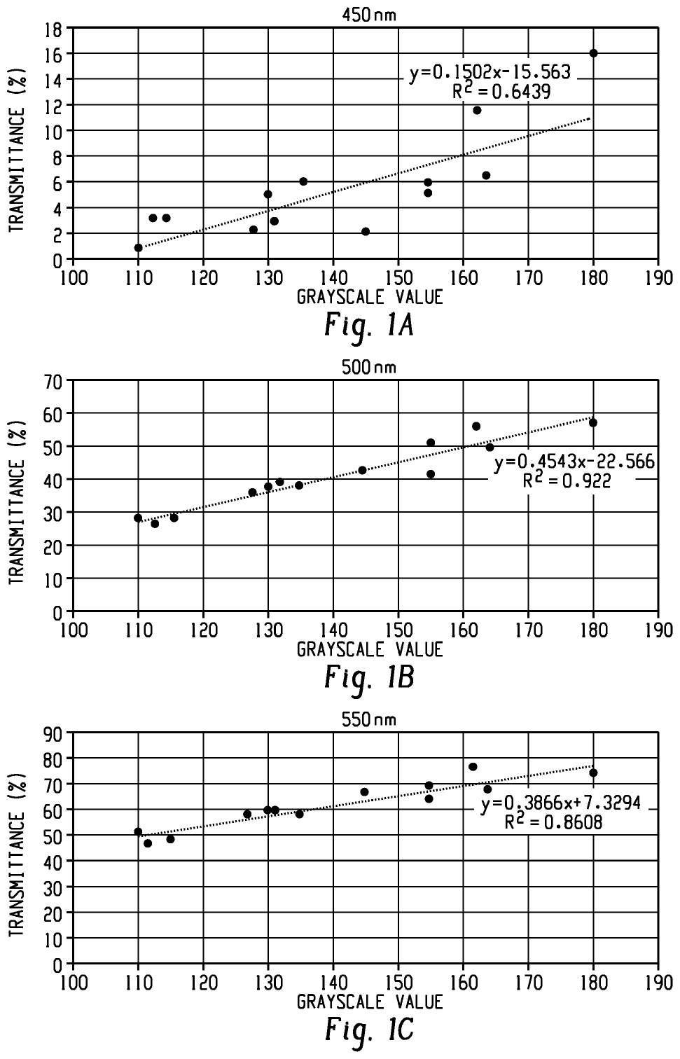

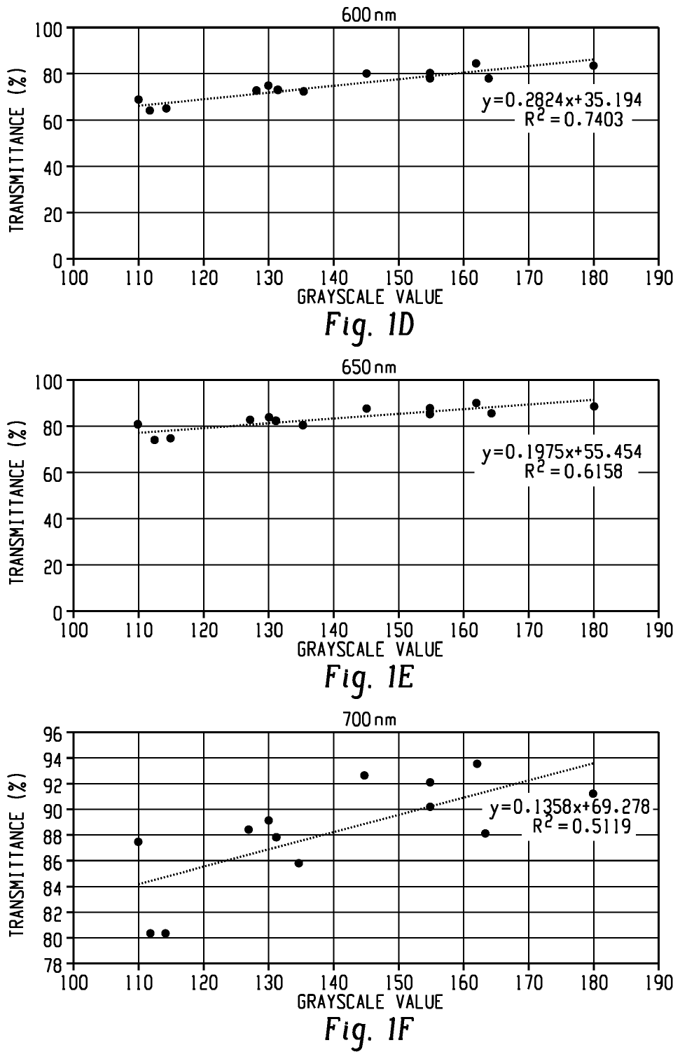

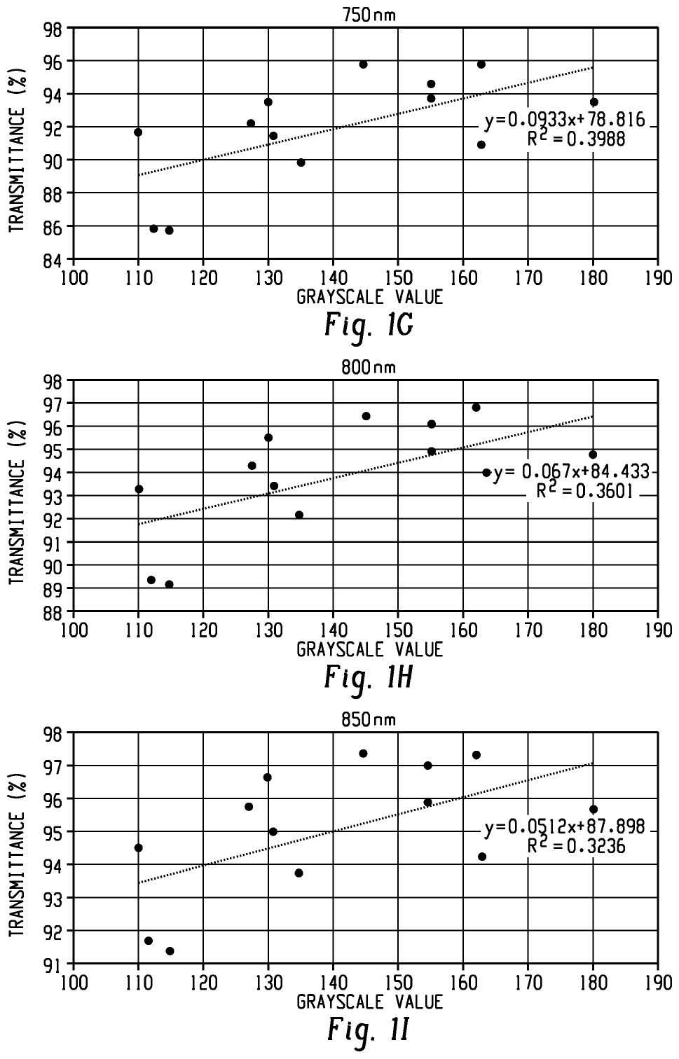

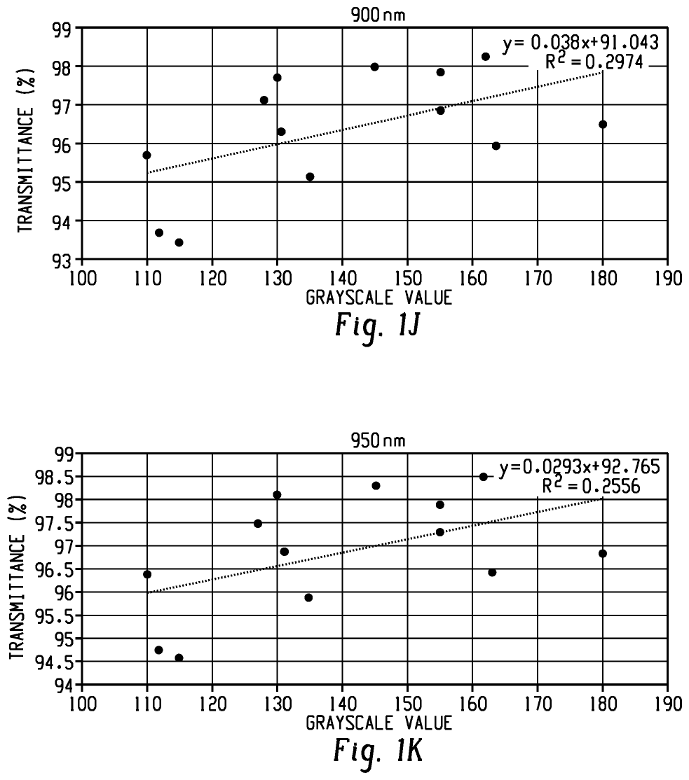

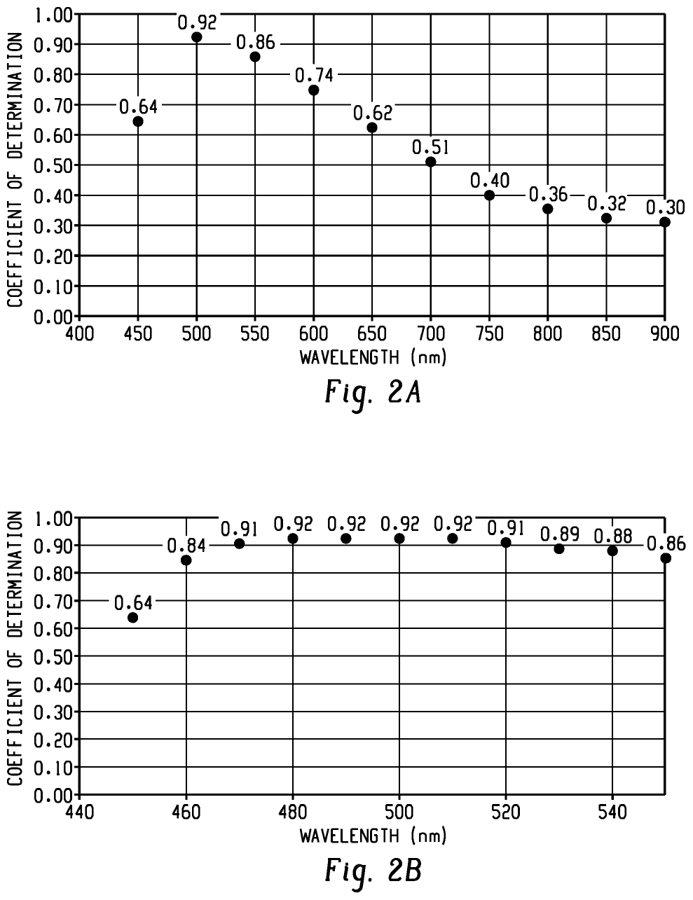

The same procedure was followed to obtain the absorbance/transmission data in FIGS. 1 and 2.

TABLE 2

| | 950 nm | | 850 nm | |
|---|---|---|---|---|
| | Transmittance | 950 nm | Transmittance | 850 nm |
| Sample | (%) | Absorbance | (%) | Absorbance |
| A | 98.63 | 0.006 | 97.77 | 0.010 |
| B | 98.03 | 0.009 | 96.88 | 0.014 |
| C | 95.34 | 0.021 | 93.17 | 0.031 |
| D | 96.33 | 0.016 | 94.69 | 0.024 |
| E | 96.48 | 0.016 | 94.61 | 0.024 |
| F | 97.36 | 0.012 | 95.66 | 0.019 |
| G | 98.13 | 0.008 | 96.68 | 0.015 |
| H | 98.07 | 0.009 | 96.74 | 0.014 |

Correlation of Absorbance/Transmittance with YI

At the concentration of 0.087 g/mL of PEI in NMP (2.175 gram in 25 mL), the observed color of the prepared solution varies from the light yellow to dark brown color. With a light path length of 1 centimeter (cm) used in the final testing protocol, the transmittance data in the Near-infrared (NIR) region (750-1000 nm) of the PEI solution is higher than 80% for all the samples, which is consistent with the high optical transparency of the PEI in this spectral region. Although the profiles of transmittance spectra of all the tested samples are largely similar, the yellowness index (YI) for the samples indicated significant sample-to-sample variation in terms of optical quality, consistent with the color variation by visual observation. Out of the eight samples tested, Sample C had the highest yellowness index value among all the samples tested and the highest absorbance at 850 nm and 950 nm of the samples tested. Sample C had a dark brown color in solution.

Table 3 shows data for the yellowness index of a color chip and solution phase for the samples tested.

TABLE 3

| | Yellowness Index | YI |
|---|---|---|
| Sample | (color chip) | Solution phase |
| A | 156 | 82.31 |
| B | 166 | 83.84 |
| C | 235 | 121.13 |
| D | 199 | 104.15 |
| E | 212 | 110.60 |
| F | 208 | 112.47 |
| G | 225 | 110.04 |
| H | 203 | 106.48 |

Correlation of Absorbance/Transmittance with Grayscale

Grayscale values for the samples were provided. The grayscale values were correlated to the absorbance (transmittance) values obtained. Linear coefficients of determination were calculated from the absorbance at wavelength range from 450 nm to 950 nm and grayscale values. Overall, the correlation was good in the visible wavelength range from 450 to 550 nm, but was poor in the higher wavelength NIR region. The coefficient of determination (about 0.92) is the highest at a wavelength of 500 nm (FIGS. 1 and 2). The results indicate the grayscale test is more related to the absorbance/transmittance in the visible wavelength region than in the NIR region. The mathematical conversion (Table 4) used in this work for the transmittance is very different from the existing yellowness index (YI) calculation. The grayscale can be predicted by determining the transmittance of a solution sample as described herein at one or more wavelengths between 400 and 800 nm, preferably one or more wavelengths between 450 and 600 nm, more preferably one or more wavelengths between 460 and 550 nm, and by multiplying the transmittance at each wavelength by the coefficient at that wavelength. Any number of wavelengths can be used.

Table 4 shows multivariate linear regression results of transmittance vs grayscale values. The predetermined prediction equation used for the prediction of grayscale in Table 4 is:

Grayscale value=(Transmittance at 460 nm*Coefficient at 460 nm)+(Transmittance at 470 nm*Coefficient at 470 nm)+ . . . +(Transmittance at 550 nm*Coefficient at 550 nm)

The coefficients are listed in Table 4.

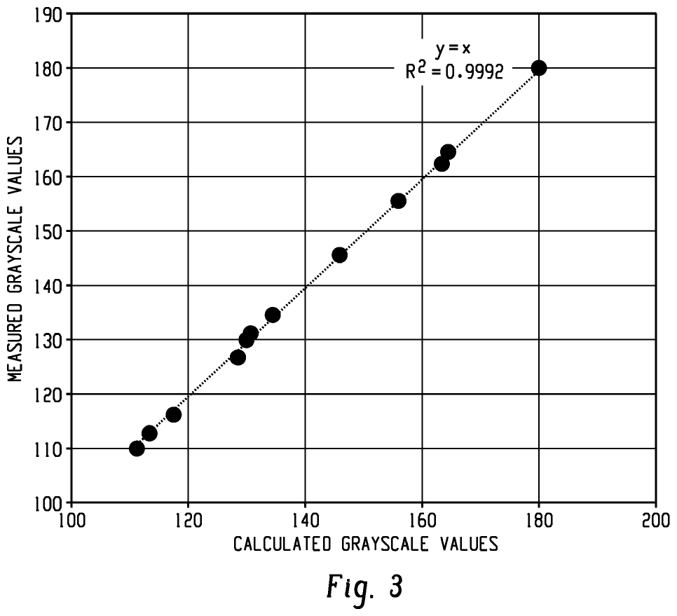

The prediction results of grayscale were compared with grayscale values provided as shown in FIG. 3.

TABLE 4
SUMMARY OUTPUT
Regression Statistics

| | |
|---|---|
| Multiple R | 0.999579068 |
| R Square | 0.999158313 |
| Adjusted R Square | 0.994949881 |
| Standard Error | 1.564881675 |
| Observations | 13 |

ANOVA

| | | | | | [symbol] gnificance |
|---|---|---|---|---|---|
| | df | SS | MS | F | [symbol] F [symbol] |
| Regression | 10 | 5814.025368 | 581.4025368 | 237.4181478 | 0.004201 |
| Residual | 2 | 4.897709313 | 2.448854657 | | |
| Total | 12 | 5818.923077 | | | |

| | | Standard | | | Lower | Upper | [symbol] ower | [symbol] pper |
|---|---|---|---|---|---|---|---|---|
| | Coefficients | Error | t Stat | P-value | 95% | 95% | 95.0 [symbol] | 95.0 [symbol] |
| Intercept | 273.51804 | 35.57680958 | 7.688099164 | 0.016500912 | 120.4434 | 426.5927 | 120.4434 | 426.5927 |
| Transmittance | 4.828108358 | 4.67621724 | 1.032481621 | 0.41034911 | −15.292 | 24.94825 | −15.292 | 24.94825 |
| at 460 nm | | | | | | | | |
| Transmittance | −21.15292591 | 14.32153872 | −1.477000924 | 0.277707561 | −82.7735 | 40.46768 | −82.7735 | 40.46768 |
| at 470 nm | | | | | | | | |
| Transmittance | 36.31044439 | 17.22649875 | 2.10782498 | 0.169589235 | −37.8092 | 110.4301 | −37.8092 | 110.4301 |
| at 480 nm | | | | | | | | |
| Transmittance | −103.3137228 | 21.53881423 | −4.796630015 | 0.040820922 | −195.988 | −10.6397 | −195.988 | −10.6397 |
| at 490 nm | | | | | | | | |
| Transmittance | 135.9334733 | 49.61465778 | 2.73978456 | 0.111396687 | −77.5412 | 349.4081 | −77.5412 | 349.4081 |
| at 500 nm | | | | | | | | |
| Transmittance | −6.704502153 | 45.30262788 | −0.14799367 | 0.895921009 | −201.626 | 188.217 | −201.626 | 188.217 |
| at 510 nm | | | | | | | | |
| Transmittance | −172.3088761 | 51.71719272 | −3.331752305 | 0.079492028 | −394.83 | 50.21224 | −394.83 | 50.21224 |
| at 520 nm | | | | | | | | |
| Transmittance | 201.9651302 | 57.92262187 | 3.48680919 | 0.073320176 | −47.2558 | 451.1861 | −47.2558 | 451.1861 |
| at 530 nm | | | | | | | | |
| Transmittance | −16.78940616 | 24.45756428 | −0.6864709 | 0.563318828 | −122.022 | 88.443 | −122.022 | 88.443 |
| at 540 nm | | | | | | | | |
| Transmittance | −60.63632934 | 20.3873286 | −2.974216511 | 0.096894465 | −148.356 | 27.08327 | −148.356 | 27.08327 |
| at 550 nm | | | | | | | | |

RESIDUAL OUTPUT

| Observation | Predicted Y | Residuals |
|---|---|---|
| 1 | 109.1431448 | 0.856855246 |
| 2 | 154.8369622 | 0.163037803 |
| 3 | 129.9020093 | 0.097990722 |
| 4 | 145.4024551 | −0.402455138 |
| 5 | 130.3838175 | 0.616182474 |
| 6 | 180.0513319 | −0.051331906 |
| 7 | 134.3038789 | 0.69612109 |
| 8 | 111.8882181 | 0.111781944 |
| 9 | 116.0268441 | −1.02684414 |
| 10 | 162.471915 | −0.471915049 |
| 11 | 163.3675381 | 0.632461874 |
| 12 | 155.03547 | −0.035470015 |
| 13 | 128.1864149 | −1.186414906 |

[symbol]

indicates data missing or illegible when filed

The methods are further illustrated by the following embodiments, which are non-limiting.

### Embodiment 1

A method for predicting the material performance of a polyimide, the method comprising: dissolving a sample of the polyimide in a solvent to form a polyimide solution; determining a transmittance of the polyimide solution at a wavelength of 400 to 800 nm, preferably at a wavelength of 450 to 600 nm to obtain a test value; inputting the test value into a predetermined prediction equation to obtain a predicted material performance value; and determining if the predicted material performance value is within a desired range of material performance.

### Embodiment 2

The method of Embodiment 1, wherein the predetermined prediction equation is determined by: obtaining a performance value for a polyimide sample; obtaining a transmittance value for the polyimide sample; calculating a mathematical relationship between the performance value and the transmittance value to obtain a predetermined prediction equation.

### Embodiment 3

The method of Embodiment 1 or 2, wherein the performance value is related to an absorbance property, a transmittance property, a color property, an optical property, an aesthetic property, or a combination comprising at least one of the foregoing.

### Embodiment 4

The method of any one or more of the preceding Embodiments, wherein the desired range of material performance is a grayscale value between 50 and 250.

### Embodiment 5

The method of any one or more of the preceding Embodiments, wherein the predetermined prediction equation is recalled from a storage medium.

### Embodiment 6

The method of any one or more of the preceding Embodiments, wherein the polyimide is a polyetherimide homopolymer, a polyetherimide copolymer, or a combination comprising at least one of the foregoing.

### Embodiment 7

The method of any one or more of the preceding Embodiments, wherein the polyetherimide copolymer is a polyetherimide sulfone.

### Embodiment 8

The method of any one or more of the preceding Embodiments, wherein the solvent is N-methyl-2-pyrrolidone (NMP), dichloromethane, chloroform, dimethyl sulfoxide (DMSO), tetrahydrofuran (THF), dimethylacetamide (DMAcAm), N,N-dimethylformamide (DMF), hexafluoroisopropanol (HFIP), cresol, or a combination comprising at least one of the foregoing.

### Embodiment 9

The method of any one or more of the preceding Embodiments, wherein the polyimide solution is at a concentration between 0.001 grams/milliliter (g/mL) to 1 g/mL, preferably 0.05 g/mL to 0.2 g/mL.

### Embodiment 10

The method of Embodiment 1, wherein the sample is 0.01 grams to 2 kilograms of the polyetherimide, preferably wherein the sample is 0.5 grams to 100 grams of the polyetherimide.

### Embodiment 11

The method of any one or more of the preceding Embodiments, further comprising rejecting the polyimide if the predicted material performance value is not within the desired range of material performance.

### Embodiment 12

A method for the manufacture of a polyimide, the method comprising manufacturing the polyimide; and predicting the material performance of the polyimide in accordance with any one or more of Embodiments 1 to 11.

### Embodiment 13

The method of Embodiment 12, further comprising continuously obtaining the sample during the manufacturing.

### Embodiment 14

The method of Embodiment 12 or 13, further comprising recycling the polyimide if the predicted material performance value is not within the desired range of material performance.

### Embodiment 15

A method of manufacturing an article from a polyimide, the method comprising: predicting the material performance of the polyimide in accordance with any one or more of Embodiments 1 to 12; and proceeding with manufacture of the predicted material performance value is within a desired range of material performance.

### Embodiment 16

The method of Embodiment 15, wherein the article is a component of an electronics article, preferably wherein the electronics article is a handheld device, more preferably wherein the article is an optical lens.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt %, or, more specifically, 5 wt % to 20 wt %”, is inclusive of the endpoints and all intermediate values of the ranges of “5 wt % to 25 wt %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through carbon of the carbonyl group.

Unless substituents are otherwise specifically indicated, each of the foregoing groups can be unsubstituted or substituted, provided that the substitution does not significantly adversely affect synthesis, stability, or use of the compound. “Substituted” means that the compound, group, or atom is substituted with at least one (e.g., 1, 2, 3, or 4) substituents instead of hydrogen, where each substituent is independently nitro (—NO₂), cyano (—CN), hydroxy (—OH), halogen, thiol (—SH), thiocyano (—SCN), C₁₋₆ alkyl, C₂₋₆ alkenyl, C₂₋₆ alkynyl, C₁₋₆ haloalkyl, C₁₋₉ alkoxy, C₁₋₆ haloalkoxy, C₃₋₁₂ cycloalkyl, C₅₋₁₈ cycloalkenyl, C₆₋₁₂ aryl, C₇₋₁₃ arylalkylene (e.g., benzyl), C₇₋₁₂ alkylarylene (e.g., toluyl), C₄₋₁₂ heterocycloalkyl, C₃₋₁₂ heteroaryl, C₁₋₆ alkyl sulfonyl (—S(═O)₂-alkyl), C₆₋₁₂ arylsulfonyl (—S(═O)₂-aryl), or tosyl (CH₃C₆H₄SO₂⁻), provided that the substituted atom's normal valence is not exceeded, and that the substitution does not significantly adversely affect the manufacture, stability, or desired property of the compound. When a compound is substituted, the indicated number of carbon atoms is the total number of carbon atoms in the compound or group, including those of any substituents.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method for predicting the material performance of a polyimide, the method comprising:
dissolving a sample of the polyimide in a solvent to form a polyimide solution;
determining a transmittance of the polyimide solution at a wavelength of 400 to 800 nm, preferably at a wavelength of 450 to 600 nm to obtain a test value;
inputting the test value into a predetermined prediction equation to obtain a predicted material performance value; and
determining if the predicted material performance value is within a desired range of material performance.

2. The method of claim 1, wherein the predetermined prediction equation is determined by:
obtaining a performance value for a polyimide sample;
obtaining a transmittance value for the polyimide sample;
calculating a mathematical relationship between the performance value and the transmittance value to obtain a predetermined prediction equation.

3. The method of claim 1, wherein the performance value is related to an absorbance property, a transmittance property, a color property, an optical property, an aesthetic property, or a combination comprising at least one of the foregoing.

4. The method of claim 1, wherein the desired range of material performance is a grayscale value between 50 and 250.

5. The method of claim 1, wherein the predetermined prediction equation is recalled from a storage medium.

6. The method of claim 1, wherein the polyimide is a polyetherimide homopolymer, a polyetherimide copolymer, or a combination comprising at least one of the foregoing.

7. The method of claim 1, wherein the polyetherimide copolymer is a polyetherimide sulfone.

8. The method of claim 1, wherein the solvent is N-methyl-2-pyrrolidone, dichloromethane, chloroform, dimethyl sulfoxide, tetrahydrofuran, dimethylacetamide, N,N-dimethylformamide, hexafluoroisopropanol, cresol, or a combination comprising at least one of the foregoing.

9. The method of claim 1, wherein the polyimide solution is at a concentration between 0.001 grams/milliliter (g/mL) to 1 g/mL, preferably 0.05 g/mL to 0.2 g/mL

10. The method of claim 1, wherein the sample is 0.01 grams to 2 kilograms of the polyetherimide, preferably wherein the sample is 0.5 grams to 100 grams of the polyetherimide.

11. The method of claim 1, further comprising rejecting the polyimide if the predicted material performance value is not within the desired range of material performance.

12. A method for the manufacture of a polyimide, the method comprising
manufacturing the polyimide; and
predicting the material performance of the polyimide in accordance with the methods of claim 1.

13. The method of claim 12, further comprising continuously obtaining the sample during the manufacturing.

14. The method of claim 12, further comprising recycling the polyimide if the predicted material performance value is not within the desired range of material performance.

15. A method of manufacturing an article from a polyimide, the method comprising:
predicting the material performance of the polyimide in accordance with the method of claim 1; and
proceeding with manufacture of the predicted material performance value is within a desired range of material performance.

16. The method of claim 15, wherein the article is a component of an electronics article, preferably wherein the electronics article is a handheld device, more preferably wherein the article is an optical lens.
